(19) United States
(12) Patent Application Publication
(10) Pub. No.: US 2011/0257670 A1
(43) Pub. Date: Oct. 20, 2011

(54) SURGICAL DEVICE FOR TISSUE PERFORATION

(76) Inventor: Gérard Scortecci, Nice (FR)

(21) Appl. No.: 13/123,826

(22) PCT Filed: Oct. 14, 2008
(86) PCT No.: PCT/EP2008/063796

§ 371 Date: Jul. 7, 2011

(51) Int. Cl. A61B 17/32

(52) U.S. Cl. 606/184

(57) ABSTRACT

A surgical device for tissue perforation (1) includes a perforation member (2), characterized in that it includes a stop (4) that presses on the outer surface of tissue (1) and is mobile relative to the perforation member, and reaction elements (3) configured to resist the movement of the stop (4) relative to the perforation member (2). The device will be used for the perforation of all types of tissue and especially bone tissues during which the withdrawal of the perforation member from the tissue is difficult.

The present invention concerns a surgical device for tissue perforation, preferably of the non-rotary type in order to avoid debris and tissues heating or rolling up.

The invention will be used for the perforation of all types of human and animal tissue and especially bone or cartilaginous tissues.

Surgical perforation devices such as existing surgical mallets equipped with punch, whether manual, mechanical, hydraulic or pneumatic, only ensure that the punch engages in this tissue. Current surgical instruments, especially non-rotary devices, encounter problems of blocking or difficulties when withdrawing of the device from the point of perforation at the end of the use, with the result that the operator has to extract the punch. With the existing devices, the operator has to pull strongly the device when the punch remains impacted and regularly blocks in the perforated bone tissue.

Moreover, these devices are without any stop pressing on the tissue. Therefore, there is no base that would enable the operator to withdraw the punch out of bone or cartilaginous tissue while remaining in the axis of perforation.

Thus, there is a need for an improved surgical device for bone or cartilaginous perforation that facilitates use and allows recovery without deforming or breaking the perforating point.

To this end, the present invention consists of a surgical device for tissue perforation that uses conventional means for perforating human or animal tissue. The device according to the invention also comprises a stop which presses on the tissue to be perforated and is mobile relative to the means of perforation, and reaction means that are designed to resist the movement of the stop.

During the perforation of tissue, the perforation means penetrate the tissue and the stop which is pressed on the outer surface of the tissue moves relative to the perforation means. This relative displacement of the stop and the perforation means acts on the reaction means which resist this movement.

Hence, the reaction means contribute to the return of the stop to its initial position relative to the perforation means in order to facilitate withdrawal of the perforation means out of the tissue.

This invention brings an improvement because the stop pressing on the outer surface of the tissue also has a centring and support function allowing more accurate control of insertion and withdrawal of the means of perforation tissue.

Other goals and advantages will appear during the description which follows of a preferred embodiment, which however is not restrictive. This invention concerns a surgical device for a tissue perforation comprising perforation means, characterized in that it comprises a stop that presses on the outer surface of the tissue and is mobile relative to the perforation means and reaction means configured to resist the movement of the stop relative to the perforation means.

According to preferred but not exhaustive alternatives, the surgical device according to the invention is such that:

the stop cooperates with the perforation means to form a seal that limits undesirable material entering between the perforation means and the stop,

the stop surrounds the perforation means,

the stop comprises an application surface peripheral to the perforation means,

the stop comprises an end-of-travel stop that limits the displacement of the perforation means relative to the stop,

the end-of-travel stop is located in the proximal part of the stop pressing on the outer surface of a tissue,

the reaction means are elastically deformable,

the reaction means are compressible,

the reaction means comprise a spring,

the reaction means are made of an elastically deformable material,

the stop is formed by the distal end of the reaction means.

The present invention concerns moreover an impulse impactor for surgical perforation of a tissue comprising the surgical device described above and impulse generating means.

The attached drawings are given as examples and are not restrictive. They only show one embodiment of the invention that will make it more easily understood.

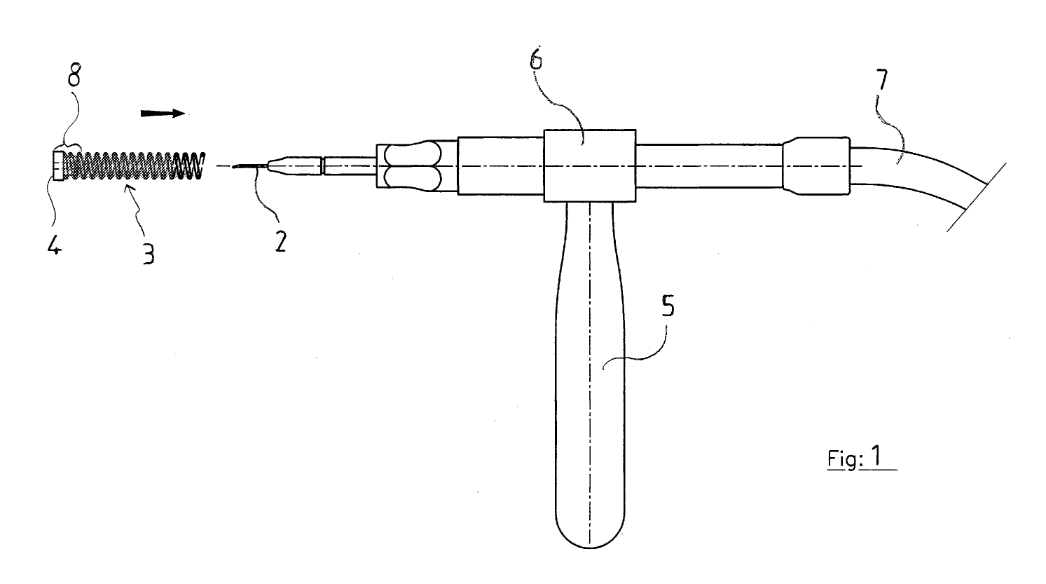

FIG. 1: side view of a device according to the invention with the reaction means and the mobile stop not yet in position.

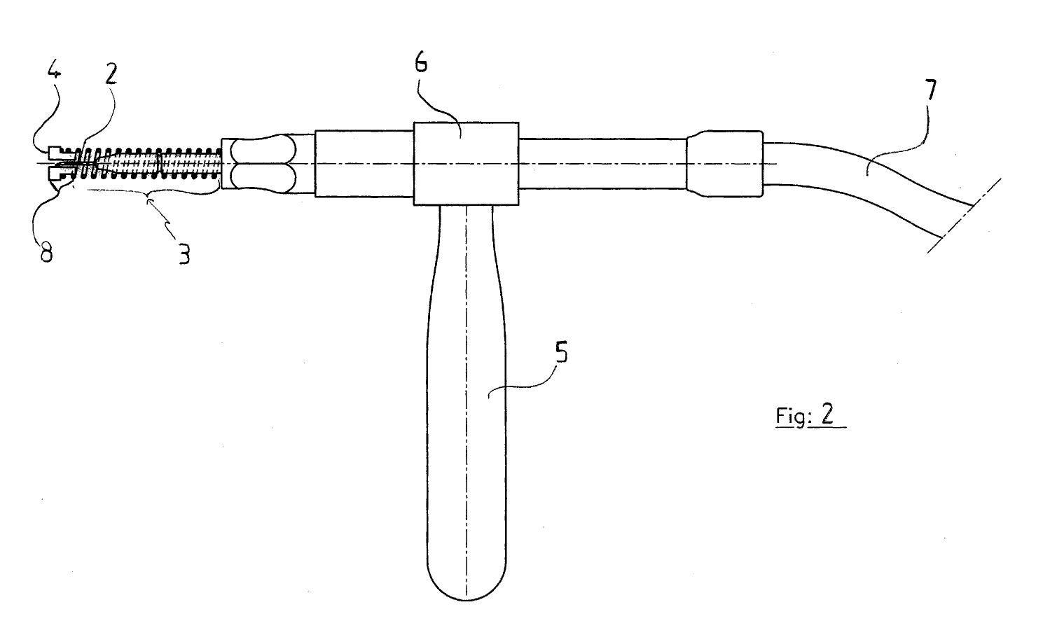

FIG. 2: side view of the device according to FIG. 1, the reaction means and the mobile stop being in place on the perforation means.

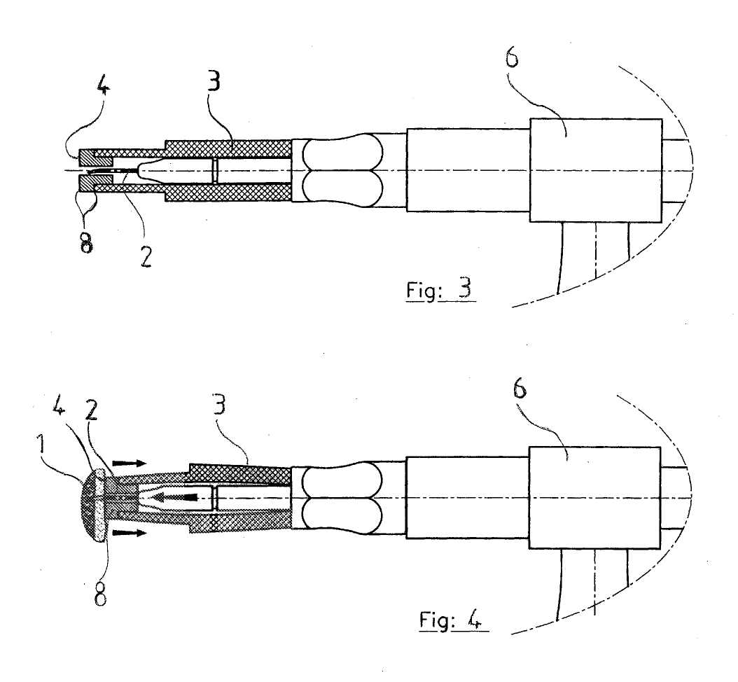

FIG. 3: side view of the device according to another embodiment.

FIG. 4: side view of the device according to FIG. 3, the perforation means perforating human or animal tissue.

The surgical device according to the invention is designed for the perforation of human or animal tissue and more precisely hard bone or cartilaginous type of tissues. The device comprises a body 6 with a handle 5 to be held by the operator. Body 6 has perforation means 2 at its distal end.

Perforation means 2 can be of various types according to the tissue to be perforated, the reason for the perforation, etc. . . . For instance, perforation means 2 are a punch or a hyper rigid probe, joined to body 6 of the device by screwing, keying or another technique for non-definitive fixing. Indeed, the perforation means 2 are generally detachable from the body 6 so as to be removed for changing, cleaning, sterilizing or discarding.

The device according to the invention comprises in addition a stop 4 pressing on the outer surface of tissue 1 to be perforated. This stop 4 is mobile relative to the perforation means 2.

The device also comprises reaction means 3 configured to resist the movement of the stop 4 relative to the perforation means 2. These reaction means 3 act as return propulsion means for the stop 4 tending to facilitate withdrawal of perforation means 2 out of tissue 1 to be perforated. Advantageously, the reaction means 3 are deformable elastically.

On the movement of the stop 4 relative to the perforation means 2 penetrating the tissue 1 to be perforated, the reaction means 3 become deformed elastically by storing energy resulting from the forward force exerted on the device. When the forward force on the device is released and becomes less than the energy stored by the reaction means 3, their elastic properties cause them to release their energy and adopt their initial configuration.

A withdrawal movement by the practitioner can be simultaneously associated with the force of reaction, in particular when there is strong resistance to punch extraction.

According to a preferred embodiment, the reaction means 3 are arranged at the level of the perforation means 2.

In a further advantageous configuration, the stop 4 is formed by the distal part of the reaction means 3. Thus, it is the distal part of the reaction means 3 that presses on the external surface of the tissue 1 to be perforated and thus constitutes the stop 4.

The stop 4 is advantageously disposed around the perforation means 2 in order to surround them. It may have a hollow cylindrical section. Moreover, the stop 4 cooperates with the perforation means 2 in a hermetic manner to limit undesirable material entering between the stop 4 and the perforation means 2. This sealing prevents any blood or debris being injected inside the device during the perforation of tissue 1. A plastic end piece with an end forming seal (by a seal or using a material such as elastomer or rubber) gives satisfaction.

According to the embodiment shown on FIGS. 1 to 4, the reaction means 3 surrounding the perforation means 2 are placed between a proximal bearing surface located on the body 6 of the device and the stop 4 pressing on the outer surface of tissue 1.

This embodiment that consists in adapting the reaction means 3 equipped with the stop 4 at the position of the perforation means 2 makes it possible to continue to use conventional perforation devices onto which the reaction means 3 and the stop 4 can be adapted.

The stop 4 can be joined by mechanical, adhesive or other means to the end of the reaction means 3.

Other embodiments can be considered wherein the reaction means 3 are laid out proximally relative to the perforation means 2 and only one stop 4 is placed at the position of the perforation means 2.

According to a first embodiment shown on FIGS. 1 and 2, the reaction means 3 that are preferably elastically deformable are of the spring type. By spring we mean an elastic part, generally but not exclusively made from quenched steel, with the property of returning to its original shape and its initial position after deformation. A stiffness of between 0.8 and 2 N/mm, in particular 1.3 N/mm, is satisfactory.

According to a second embodiment shown on FIGS. 3 and 4, the reaction means 3 can be formed by a tubular part, containing elastically deformable materials. Various types of polymers with elastic properties can be used, in particular elastomers.

Any other form of reaction means, in particular gas compression in a chamber, elastic deformation of a part in flexure are within the context of the invention.

Preferably, the device according to the invention comprises an end-of-travel stop 8 designed to limit displacements of the stop 4 relative to the perforation means 2.

According to this embodiment, the end-of-travel stop 8 is formed at the level of the stop 4 in the distal part of the reaction means 3. This end-of-travel stop 3 is made of a rigid material but does not have an elastic property to withstand the pressure and thus block the movement of the perforation means 2 relative to the stop 4.

The presence of this end-of-travel stop 8 increases the safety of the device according to the invention. Indeed, when the operator inserts the perforation means 2 into tissue 1, it will not perforate tissue 1 too deeply.

Another advantage is that the thickness of the end-of-travel stop 8 can be modulated so that the operator can change the depth of perforation.

Similarly, the resistance of the reaction means 3 to the movement of the stop 4 relative to the perforation means 2 can also be controlled by the operator. Thus, when perforating softer tissue, the resistance of the reaction means 3 will be regulated so as to be lower as the withdrawal of the perforation means 2 from the tissue 1 is already easy. On the other hand, when perforating bone tissue, there will be high resistance of the reaction means 3 thus largely facilitating the withdrawal of the perforation means 2 out of the bone tissue where conventional perforation means traditionally block.

Below is a description of the method for using the surgical device according to the invention.

The operator places in the distal part of the body 6 of the device the perforation means 2 of the punch type.

The stop 4 is applied to the outer surface of tissue 1 to be perforated, the operator then applies pressure on handle 5 of the body 6 of the device so as to insert perforation means 2 into tissue 1 to be perforated. On this insertion of the perforation means 2 into the tissue 1 to be perforated, the stop 4 moves relative to the perforation means 2 and the reaction means 3 oppose this displacement by deforming elastically.

When the perforation of tissue 1 has been completed, either because the reaction means 3 stop on the end-of-travel stop 8 or the operator considers that perforation means 2 have been sufficiently inserted in tissue 1, he slackens the pressure on handle 5 of the device, and the reaction means 3 having stored the forward force energy on insertion will release this energy and will contribute to moving the stop 4 relative to the perforation means 2 so as to withdraw the perforation means 2 out of the tissue 1.

It is an advantage to be able to envisage a bearing surface for the stop 4 on a relatively large outer surface of the tissue 1 as this allows centring of the perforation means 2 during the perforation of the tissue 1, as well as a distribution of the forces applied by the reaction means 3 when they release their energy and push back the device so as to extract the perforation means 2 out of the tissue 1.

The device according to the invention is preferably of an impact type designed to generate impulses programmed beforehand in terms of both a force and cycle, to enable tissue penetration by impulses.

The body 6 of the device is advantageously connected to an end piece 7 connecting to the energy source such as an air, mechanical water or pneumatic pressure micro-motor.

All the device according to the invention can be sterilised or be sterile for mono-usage.

The device according to the invention can be used to perforate various types of tissue in various parts of the human or animal body such as bone tissue in the buccal cavity, the orofacial area, the hand, the spinal column or the foot, etc.

The device is used for the treatment of chronic osseous pathologies by transparietal mechanotherapy.

The device according to the invention can also be used to convey medication and/or molecules dedicated to local treatment of osteoporosis.

For instance, parodontisis and osteoporosis of the jaw bone can be treated effectively with the invention.

### REFERENCES

1. Tissue
2. Perforation means
3. Reaction means
4. Stop
5. Handle
6. Body
7. Connecting end piece
8. End-of-travel stop

1. Surgical device for a tissue (1) perforation comprising perforation means (2) characterized in that it comprises a stop (4) that presses on the outer surface of tissue (1) and is mobile relative to perforation means (2), and reaction means (3) configured to resist the movement of the stop (4) relative to the perforation means.

2. Device according to claim 1 wherein the stop (4) cooperates with the perforation means (2) to form a seal that limits undesirable material entering between the perforation means (2) and the stop (4).

3. Device according to claim 1 wherein the stop (4) surrounds the perforation means (2).

4. Device according to claim 1 wherein the stop (4) comprises an application surface peripheral to the perforation means (2).

5. Device according to claim 1 wherein the stop (4) comprises an end-of-travel stop (8) that limits the displacement of the perforation means (2) relative to the stop (4).

6. Device according to claim 5 wherein the end-of-travel stop (8) is located in the proximal part of the stop (4) pressing on the outer surface of a tissue (1).

7. Device according to claim 1 wherein the reaction means (3) are elastically deformable.

8. Device according to claim 1 wherein the reaction means (3) are compressible.

9. Device according to claim 1 wherein the reaction means (3) comprise a spring.

10. Device according to claim 1 wherein the reaction means (3) are made of an elastically deformable material.

11. Device according to claim 1 wherein the stop (4) is formed by the distal end of the reaction means (3).

12. Impulse impactor for surgical perforation of a tissue comprising the surgical device according to claim 1 and impulse generating means.
